(19) United States
(12) Patent Application Publication
(10) Pub. No.: US 2016/0030124 A1
(43) Pub. Date: Feb. 4, 2016

(54) SURGICAL TOOL HOLDING DEVICE, ENDOSCOPE, AND MEDICAL SYSTEM

(71) Applicant: OLYMPUS CORPORATION, Tokyo (JP)

(72) Inventors: Kosuke KISHI, Tokyo (JP); Toshihiro YOSHII, Tokyo (JP)

(73) Assignee: OLYMPUS CORPORATION, Tokyo (JP)

(21) Appl. No.: 14/819,109

(22) Filed: Aug. 5, 2015

Related U.S. Application Data
(63) Continuation of application No. PCT/JP2014/054987, filed on Feb. 21, 2014.
(60) Provisional application No. 61/768,731, filed on Feb. 25, 2013.

(51) Int. Cl. A61B 19/00; A61B 1/018; A61B 1/00

(52) U.S. Cl. CPC A61B 19/26; A61B 1/00133; A61B 1/018; A61B 1/00149; A61B 2017/00017

(57) ABSTRACT

A surgical tool holding device that holds a plurality of surgical tools includes: a flexible insertion section having a distal end and a proximal end and configured such that a surgical tool channel into which the plurality of surgical tools are inserted is formed; a driving section configured to advance and retract each of the plurality of surgical tools inside the surgical tool channel; an operating section configured to manipulate the plurality of surgical tools; and a controller configured to control an amount of protrusion of the surgical tool protruding from a distal end of the surgical tool channel, from the surgical tool channel.

This application is a continuation application based on a PCT International Application No. PCT/JP2014/054987, filed on Feb. 21, 2014, whose priority is claimed on U.S. Patent Provisional Application No. 61/768,731, filed on Feb. 25, 2013. The content of the PCT International Application and the US patent Provisional application are incorporated herein by reference.

### BACKGROUND OF THE INVENTION

1. Field of the Invention

The present invention relates to a surgical tool holding device, an endoscope, and a medical system.

2. Description of Related Art

Conventionally, in medical treatments using endoscopes, systems in which medical treatments are performed while switching a plurality of surgical tools in the endoscopes have been known. For example, Japanese Unexamined Patent Application, First Publication No. 2005-080983 discloses an endoscopic treatment system on which a plurality of surgical tools are mounted.

When a surgical tool inserted into a surgical tool channel of an endoscope is replaced with another surgical tool, it is necessary to remove the surgical tool completely from the surgical tool channel and insert the other surgical tool into the surgical tool channel.

An endoscope disclosed in Japanese Unexamined Patent Application, First Publication No. 2005-080983 includes a surgical tool channel with a large diameter into which a plurality of surgical tools can be collectively inserted.

An endoscope disclosed in Japanese Unexamined Patent Application, First Publication No. 2006-087474 includes an electromotive mechanism that removes a surgical tool from a surgical tool channel at the time of replacement and inserts another surgical tool into the surgical tool channel.

An endoscope disclosed in Japanese Unexamined Patent Application, First Publication No. 2005-204995 includes a plurality of surgical tool insertion channels.

In a surgical tool including a plurality of joints at a distal end of the surgical tool, the distance between flexural centers of the respective joints is a substantial rigid length. When the plurality of joints are held in a rectilinear form, rectilinear sections formed by the respective joints correspond to the substantial rigid length.

### SUMMARY OF THE INVENTION

According to a first aspect of the present invention, a surgical tool holding device that holds a plurality of surgical tools includes: a flexible insertion section having a distal end and a proximal end, and being configured such that a surgical tool channel into which the plurality of surgical tools are inserted is formed; a driving section configured to advance and retract each of the plurality of surgical tools inside the surgical tool channel; an operating section configured to manipulate the plurality of surgical tools; and a controller configured to control an amount of protrusion of the surgical tool protruding from a distal end of the surgical tool channel, from the surgical tool channel. The insertion section includes: a distal end tube section configured to be one conduit line including an opening on a side of the distal end of the insertion section; a first proximal end tube section configured to be one conduit line including an opening on a side of the proximal end of the insertion section; a second proximal end tube section configured to be one conduit line including an opening on the side of the proximal end of the insertion section; and a connecting section in which the distal end tube section, the first proximal end tube section, and the second proximal end tube section are connected and disposed inside the insertion section when the distal end tube section, the first proximal end tube section, and the second proximal end tube section communicate with each other.

According to a second aspect of the present invention, in the surgical tool holding device according to the first aspect, the insertion section may further include: a flexible tube section; and a bending section configured to be disposed on the side of the distal end of the insertion section, connected with the flexible tube section, and operated to perform a bending operation. The connecting section may be disposed inside the flexible tube section and near a boundary between the flexible tube section and the bending section.

According to a third aspect of the present invention, in the surgical tool holding device according to the first or the second aspect, the driving section may hold a part of a section of the surgical tool extended from the side of the proximal end of the surgical tool channel to an outside of the insertion section, and may advance and retract a section of the surgical tool inserted into the surgical tool channel in a conduit line central axis direction of the surgical tool channel by advancing and retracting the held section of the surgical tool.

According to a fourth aspect of the present invention, in the surgical tool holding device according to the first or the second aspect, the driving section may include: a first driving section configured to advance and retract the surgical tool inserted into the first proximal end tube section only by a length between a distal end of the distal end tube section and the connecting section; a second driving section configured to advance and retract the surgical tool inserted into the second proximal end tube section only by the length between the distal end of the distal end tube section and the connecting section; and a linkage section configured to cause the first and second driving sections to cooperatively operate such that the surgical tool inserted into the second proximal end tube section is drawn and held into the second proximal end tube section just before the connecting section when the surgical tool inserted into the first proximal end tube section is located inside the distal end tube section and such that the surgical tool inserted into the first proximal end tube section is drawn and held into a side of the first proximal end tube section just before the connecting section when the surgical tool inserted into the second proximal end tube section is located inside the distal end tube section.

According to a fifth aspect of the present invention, in the surgical tool holding device according to any one of the first aspect to the fourth aspect, a shape of a cross-section perpendicular to a central axis of the distal end tube section, a shape of a cross-section perpendicular to a central axis of the first proximal end tube section, and a shape of a cross-section perpendicular to a central axis of the second proximal end tube section may be substantially the same. An area of the cross-section perpendicular to the central axis of the distal end tube section, an area of the cross-section perpendicular to the central axis of the first proximal end tube section, and an area of the cross-section perpendicular to the central axis of the second proximal end tube section may be substantially the same.

According to a sixth aspect of the present invention, in the surgical tool holding device according to the fifth aspect, each of the plurality of the surgical tools may include a treatment section configured to perform a treatment on a treatment target and an elongated section configured to be coupled with the treatment section and inserted into the surgical tool channel. The distal end tube section may be a tube with a dimension in which the treatment section and the elongated section of only one of the plurality of surgical tools are insertable and the treatment sections and the elongated sections of two or more of the plurality of surgical tools conflict with each other when the treatment sections and the elongated sections are simultaneously inserted.

According to a seventh aspect of the present invention, in the surgical tool holding device according to any one of the first aspect to the sixth aspect, the plurality of surgical tools may include a first surgical tool and a second surgical tool. The controller may record an amount of protrusion of the first surgical tool protruding from the distal end of the surgical tool channel and restrict an amount of protrusion of the second surgical tool protruding from the surgical tool channel so that the second surgical tool protrudes only up to the recorded amount of protrusion when the first surgical tool protruding from the distal end is drawn and the second surgical tool is caused to protrude from the distal end of the surgical tool channel.

According to an eighth aspect of the present invention, an endoscope includes the surgical tool holding device according to any one of the first aspect to the seventh aspect of the present invention; and an imaging means configured in the insertion section and configured to image the treatment target.

According to a ninth aspect of the present invention, a medical system includes the surgical tool holding device according to any one of the first aspect to the seventh aspect; a plurality of surgical tools configured to perform a treatment on a treatment target; and a manipulator configured to operate the surgical tool holding device and the plurality of surgical tools. Each of the plurality of surgical tools includes a treatment section configured to perform the treatment on the treatment target; an elongated section configured to be coupled with the treatment section and inserted into the surgical tool channel; a joint section configured to couple the treatment section with the elongated section and change an orientation of the treatment section with respect to the elongated section; and a wire configured to be disposed inside the elongated section and transmit a force to operate the joint section. The manipulator includes a master manipulator configured to receive an operation input from an operator; a slave manipulator configured to be electrically connected to the master manipulator and operate the wire in accordance with the operation input to the master manipulator; and a controller configured to be electrically connected to the master manipulator and the slave manipulator and control an orientation of the joint section. When the joint section is extended from the distal end tube conduit and a halt command of the surgical tool including the joint section is performed on the manipulator, the controller generates a signal for manipulating the wire so that the joint section is relaxed and outputs the signal to the slave manipulator. When the joint section is relaxed in response to the signal, the controller outputs, to the slave manipulator, a signal for drawing the treatment section and the elongated section until the treatment section is located inside the first or second proximal end tube section.

### BRIEF DESCRIPTION OF DRAWINGS

FIG. 1 is an overall diagram illustrating a surgical tool holding device and a medical system according to a first embodiment of the present invention.

FIG. 2 is a schematic diagram illustrating the surgical tool holding device according to the first embodiment of the present invention.

FIG. 3 is a schematic diagram illustrating an example of a surgical tool mounted on the surgical tool holding device according to the first embodiment of the present invention.

FIG. 4 is a schematic diagram illustrating a state when the surgical tool holding device according to the first embodiment of the present invention is used.

FIG. 5 is a schematic diagram illustrating one process when the surgical tool holding device according to the first embodiment of the present invention is used.

FIG. 6 is a schematic diagram illustrating one process when the surgical tool holding device according to the first embodiment of the present invention is used.

FIG. 7 is a schematic diagram illustrating one process when the surgical tool holding device according to the first embodiment of the present invention is used.

FIG. 8 is a schematic diagram illustrating a part of a surgical tool holding device according to a second embodiment of the present invention.

FIG. 9 is a schematic diagram illustrating a part of a surgical tool holding device according to a third embodiment of the present invention.

FIG. 10 is a schematic diagram illustrating a part of a surgical tool holding device according to a fourth embodiment of the present invention.

FIG. 11 is a schematic diagram illustrating a part of a surgical tool holding device according to a fifth embodiment of the present invention.

FIG. 12 is a schematic diagram illustrating a part of a surgical tool holding device according to a sixth embodiment of the present invention.

FIG. 13 is a schematically expanded view illustrating a part of the surgical tool holding device according to the sixth embodiment of the present invention.

FIG. 14 is a schematic diagram illustrating a part of a surgical tool holding device according to a seventh embodiment of the present invention.

FIG. 15 is a flowchart for describing a process of replacing surgical tools in the surgical tool holding device according to the first embodiment of the present invention.

### DETAILED DESCRIPTION OF THE INVENTION

### First Embodiment

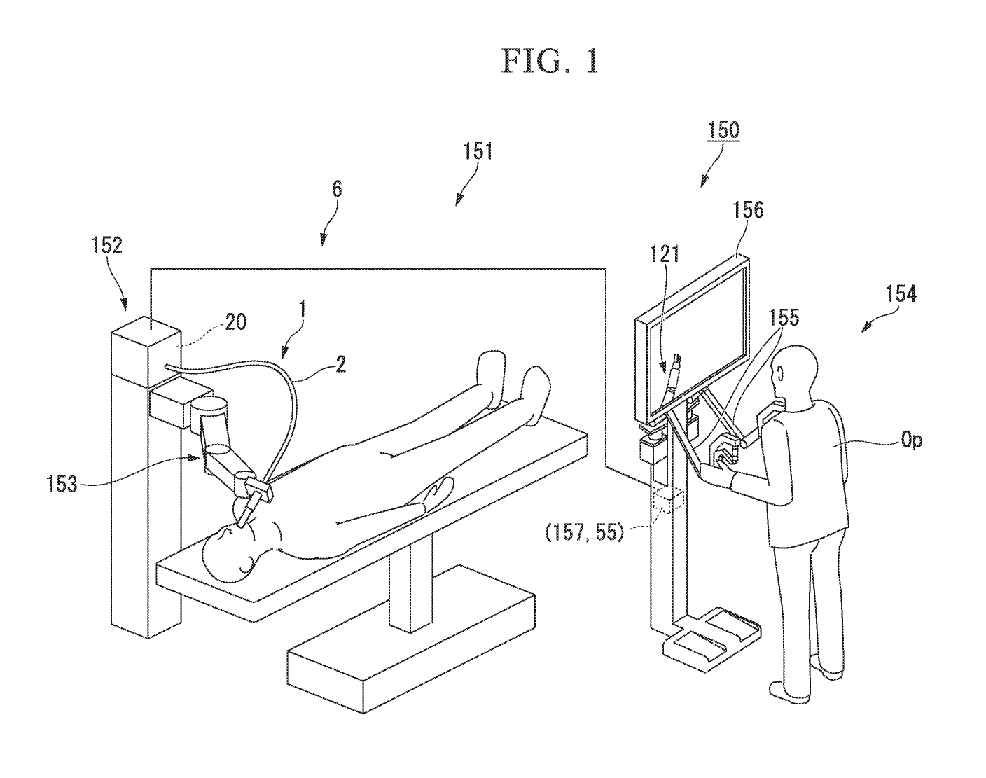

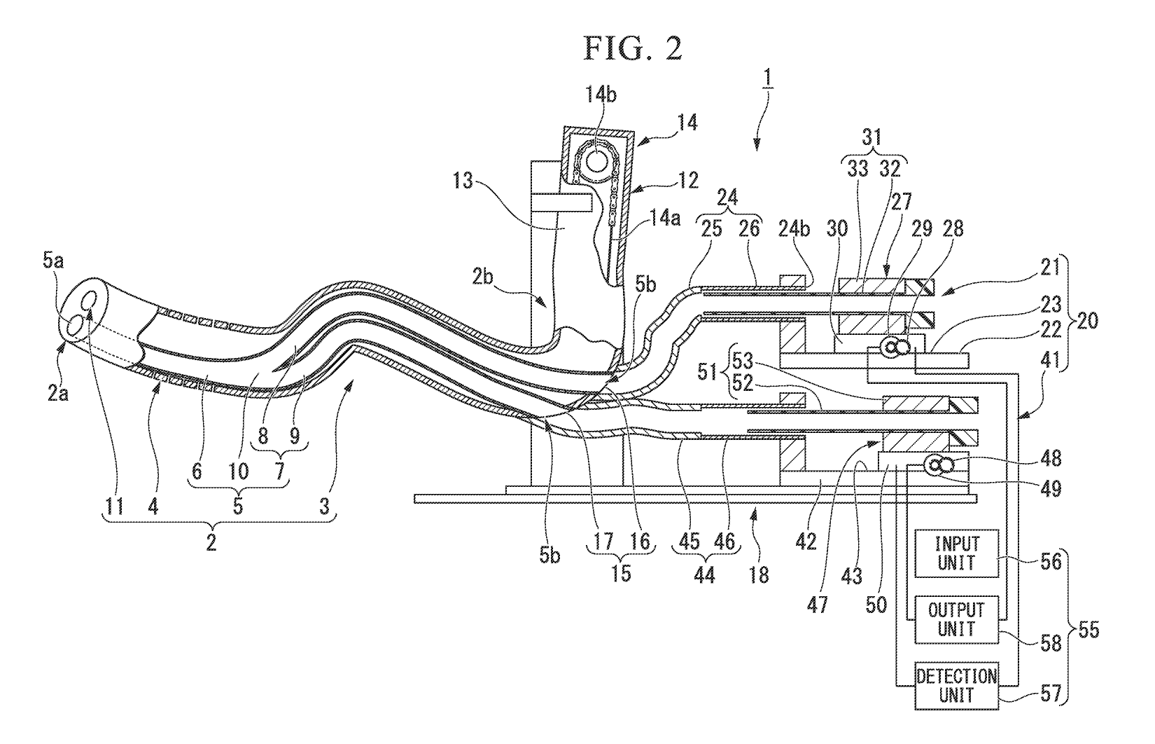

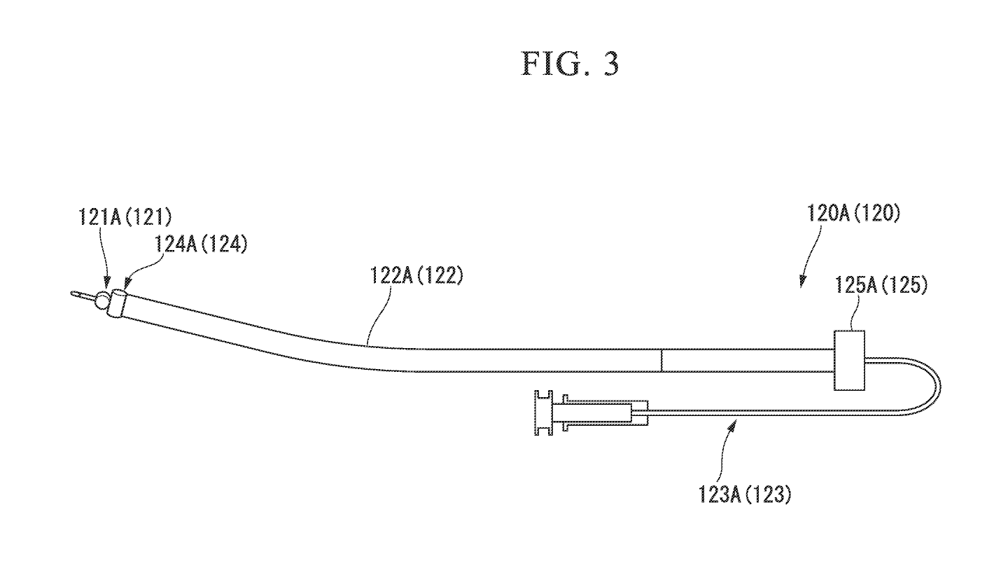

A first embodiment of a surgical tool holding device, an endoscope, and a medical system according to the present invention will be described. FIG. 1 is an overall diagram illustrating the surgical tool holding device 1 and the medical system 150 according to the first embodiment of the present invention. FIG. 2 is a schematic diagram illustrating the surgical tool holding device 1 according to the first embodiment of the present invention. FIG. 3 is a schematic diagram illustrating an example of a surgical tool 120 mounted on the surgical tool holding device 1 according to the first embodiment of the present invention. FIG. 2 is a schematically simplified diagram of the surgical tool holding device 1 illustrated in FIG. 1.

A medical system 150 (see FIG. 1) according to the present embodiment includes a surgical tool 120 (see FIG. 3), a surgical tool holding device 1 (see FIG. 2), and a manipulator 151. The surgical tool 120 performs treatment on a biological tissue. The surgical tool holding device 1 holds the surgical tool 120. The manipulator 151 operates the surgical tool 120 and the surgical tool holding device 1.

First, the configuration of the surgical tool 120 will be described. As illustrated in FIG. 3, the surgical tool 120 mounted on the surgical tool holding device 1 may be a known surgical tool (a forceps, a knife, a brush, or the like) for an endoscope used along with, for example, a flexible endoscope.

The surgical tool 120 according to the present embodiment includes a treatment section 121, a joint section 124, an elongated section 122, a mounting section 123, and an adapter section 125. The treatment section 121 performs treatment on a treatment target tissue. The elongated section 122 is coupled with the joint section 124. The joint section 124 is coupled with the treatment section 121. The mounting section 123 is coupled with the elongated section 122 and can be mounted on a movement section 27 or 47 to be described below. The adapter section 125 includes actuators operating the treatment section 121 and the joint section 124.

The adapter section 125 is disposed to curve the joint section 124 via a bending wire (not illustrated) disposed inside the elongated section 122. The adapter section 125 according to the present embodiment is electrically connected to a slave manipulator 152 to be described below. Also, the adapter section 125 operates the treatment section 121 and the joint section 124 according to signals sent from the slave manipulator 152.

Next, the configuration of the surgical tool holding device 1 will be described.

As illustrated in FIG. 2, the surgical tool holding device 1 according to the present embodiment includes an elongated insertion section 2, an operating section 12, and a driving section 20. The insertion section 2 includes a distal end 2a and a proximal end 2b. The operating section 12 is provided at the proximal end of the insertion section 2. The driving section 20 operates the surgical tool 120. The surgical tool holding device 1 according to the present embodiment includes a base 18 on which the operating section 12 and the driving section 20 are mounted.

As illustrated in FIG. 1, the insertion section 2 of the surgical tool holding device 1 includes a flexible tube section 3, a bending section 4, a channel tube 5, and an imaging means 11. The flexible tube section 3 is inserted into a body. The bending section 4 is disposed on the distal end side of the flexible tube section 3 and is coupled with the flexible tube section 3. The channel tube 5 is disposed inside the flexible tube section 3 and the bending section 4. The imaging means 11 is disposed on the distal end side of the bending section 4.

The flexible tube section 3 is an elongated member with a flexible tubular shape formed of a tube made of a resin, a tube woven out of a metal wire, or the like.

The bending section 4 is a flexible tube that performs a curving operation by driving an electric pulling means 14 provided in the operating section 12 and configured for a curving manipulation to be described below. The configuration of the bending section 4 may be a known configuration applicable to a flexible endoscopic device. For example, when a bending manipulation wire 14a is pulled in accordance with an operation of the electric pulling means 14, the bending section 4 performs the curving operation in two directions or four directions. The bending manipulation wire 14a passes through the insides of the bending section 4 and the flexible tube section 3 and is connected to the electric pulling means 14 of the operating section 12. The bending section 4 may be manipulated by an angle knob for manual manipulation instead of the electric pulling means 14.

The channel tube 5 is a flexible tubular member that is branched into two sections in a part of the inside of the insertion section 2. The channel tube 5 includes a distal end tube section 6 and a proximal end tube section 7 (a first proximal end tube section 8 and a second proximal end tube section 9). The distal end tube section 6 is one conduit line disposed on the side of the distal end 2a of the insertion section 2. The first proximal end tube section 8 and the second proximal end tube section 9 are two conduit lines disposed on the side of the proximal end 2b of the insertion section 2. The two sections into which the channel tube 5 is branched are connected when the distal end tube section 6 and the proximal end tube section 7 communicate with each other, and are a connecting section 10 between the distal end tube section 6 and the proximal end tube section 7.

The first proximal end tube section 8 and the second proximal end tube section 9 each has an opening at a proximal end 5b of the channel tube 5. The openings at the proximal end 5b of the channel tube 5 are provided in the operating section 12, and are connected to a surgical tool insertion port 15 to be described below which is disposed in the operating section 12.

A distal end 5a of the channel tube 5 is fixed to the distal end 2a of the insertion section 2 and a proximal end 5b is provided in the operating section 12. The connecting section 10 in the channel tube 5 is disposed inside the flexible tube section 3 and in the vicinity of the boundary between the flexible tube section 3 and the bending section 4. Since the channel tube 5 is one conduit line inside the bending section 4, the bending section 4 is flexibly bending more easily than a case when two conduit lines are disposed inside the bending section 4.

A middle section of the channel tube 5 may be coupled with the inner surface of the insertion section 2 at several sections or may not be coupled with the insertion section 2. Since the channel tube 5 is disposed sufficiently loosely to be movable inside the insertion section 2, high flexibility of the flexible tube section 3 and the bending section 4 can be maintained.

The distal end tube section 6 of the channel tube 5 has a circular cross-section perpendicular to a central axis of the distal end tube section 6 and has a hollow. The inner diameter dimension of the distal end tube section 6 is a size in which the treatment section 121 and the elongated section 122 in the surgical tool 120 illustrated in FIG. 3 are capable of being advanced and retracted freely along the central axis of the distal end tube section 6 and the treatment section 121 and the elongated section 122 are capable of being rotated using the central axis of the distal end tube section 6 as a rotational center. The shape and area of a cross-section perpendicular to the central axis of each of the first proximal end tube section 8 and the second proximal end tube section 9 are substantially the same as the shape and area of the cross-section perpendicular to the central axis of the distal end tube section 6. That is, the shape of the cross-section perpendicular to the central axis of the distal end tube section 6, the shape of the cross-section perpendicular to the central axis of the first proximal end tube section 8, and the shape of the cross-section perpendicular to the central axis of the second proximal end tube section 9 are substantially the same. The area of the cross-section perpendicular to the central axis of the distal end tube section 6, the area of the cross-section perpendicular to the central axis of the first proximal end tube section 8, and the area of the cross-section perpendicular to the central axis of the second proximal end tube section 9 are substantially the same.

In the connecting section 10, the distal end tube section 6, the first proximal end tube section 8, and the second proximal end tube section 9 are connected to each other to be disposed in a Y form. For example, an angle formed between the central axis of the first proximal end tube section 8 and the central axis of the second proximal end tube section 9 may be less than 90 degrees, an angle formed between the central axis of the first proximal end tube section 8 and the central axis of the distal end tube section 6 may be equal to or greater than 90 degrees, and an angle formed between the central axis of the second proximal end tube section 9 and the central axis of the distal end tube section 6 may be equal to or greater than 90 degrees. In this case, the surgical tool 120 is capable of being smoothly inserted into the distal end tube section 6 from the proximal end tube section 7 via the connecting section 10. The connecting state between the distal end tube section 6, the first proximal end tube section 8, and the second proximal end tube section 9 is not limited to the Y form.

An inner cavity of the channel tube 5 is a surgical tool channel which allows the surgical tool 120 to pass through. The inner diameter of the distal end tube section 6 may not necessarily be a size in which the treatment sections 121 and the elongated sections 122 of the two surgical tools 120 are capable of being inserted simultaneously. The inner diameter of the distal end tube section 6 is a size in which the treatment section 121 and the elongated section 122 of one surgical tool 120 is capable of being inserted and is a size in which the treatment sections 121 and the elongated sections 122 of the two surgical tools 120 are not capable of being inserted simultaneously. That is, the inner diameter dimension of the distal end tube section 6 is a dimension in which a mutual conflict occurs when the treatment sections 121 and the elongated sections 122 of the two surgical tools are inserted simultaneously. Therefore, the degree of serpentine movement of the elongated section 122 inside the distal end tube section 6 is capable of being suppressed. The distal end tube section 6 has the size of the inner diameter so as to allow the treatment section 121 and the elongated section 122 of only one surgical tool 120 to be capable of passing through the distal end tube section 6. Thus, when an operator advances and retracts the elongated section 122 along the center axis line of the channel tube 5, the operator can efficiently transmit the amount of force applied from the side of the operating section 12 to the treatment section 121.

A material of the channel tube 5 is preferably a material with low frictional resistance against the treatment section 121 and the elongated section 122 of the surgical tool 120. The inner surface of the channel tube 5 may be a surface subjected to a surface finishing process of reducing frictional resistance against the treatment section 121 and the elongated section 122.

The imaging means 11 is provided in the insertion section 2 for the purpose of imaging a treatment target tissue inside a body and the surgical tool 120 mounted on the surgical tool holding device 1. According to the present embodiment, the surgical tool holding device 1 has a function of an endoscope in that visual observation is capable of being performed. The configuration of the imaging means 11 is not particularly limited.

The operating section 12 includes a main body 13, the electric pulling means 14, and a driving section 20. The main body 13 is fixed to a proximal end of the flexible tube section 3. The electric pulling means 14 is provided in the main body 13 and pulls the bending manipulation wire 14a. The driving section 20 is provided to move the surgical tool 120 mounted on the surgical tool holding device 1 and holds the surgical tool 120. The bending manipulation wire 14a connected to the above-described bending section 4 is rolled around a pulley or a sprocket 14b. The electric pulling means 14 is provided for the purpose of performing rotation manipulation on the pulley or the sprocket 14b using a rotation shaft of the pulley or the sprocket 14b as a rotational center. A knob used to manually pull the bending manipulation wire 14a may be provided instead of the electric pulling means 14 of the operating section 12. A known mechanism such as a rack-and-pinion mechanism pulling the bending manipulation wire 14a may be provided.

Inside the main body 13, the channel tube 5 extends from the flexible tube section 3. The surgical tool insertion port 15 communicating with an opening on the side of the proximal end of each channel tube 5 is provided in the main body 13. One surgical tool insertion port 15 is provided for each channel tube 5 (the first proximal end tube section 8 and the second proximal end tube section 9) branched into a plurality of sections on the side of the proximal end of the channel tube 5. According to the present embodiment, two surgical tool insertion ports 15 (a first surgical tool insertion port 16 and a second surgical tool insertion port 17) are provided in the operating section 12. The surgical tool insertion port 15 has an inner diameter in which the treatment section 121 and the elongated section 122 of the surgical tool 120 to be described below are capable of being inserted. The surgical tool insertion port 15 may be disposed in the flexible tube section 3 rather than the operating section 12.

The driving section 20 includes a first driving section 21, a second driving section 41, and a linkage section 55. The first driving section 21 moves the surgical tool 120 inserted into a first port (the first surgical tool insertion port 16 in the present embodiment) between the two surgical tool insertion ports 15. The second driving section 41 moves the surgical tool 120 inserted into a second port (the second surgical tool insertion port 17 in the present embodiment) between the two surgical tool insertion ports 15. The linkage section 55 causes the first driving section 21 and the second driving section 41 to operate cooperatively.

The first driving section 21 includes a base section 22 and a movement section 27. The base section 22 is mounted on the base 18 detachably. The movement section 27 is advanced toward and retracted from the base section 22. The first driving section 21 operates the surgical tool 120 inserted into the first proximal end tube section 8 such that the surgical tool 120 is advanced and retracted by a length which is restricted by a length between the distal end of the distal end tube section 6 and the connecting section 10.

The base section 22 includes a rack 23 with a rectilinear shape and a connecting conduit line 24 communicating with the surgical tool insertion port 15.

The connecting conduit line 24 includes a tube 25 and a tube 26. The tube 25 is a flexible tube communicating with the surgical tool insertion port 15. The tube 26 is a rigid tube of which one end is connected to the flexible tube 25 and which is fixed to the base section 22. The elongated section 122 of the surgical tool 120 illustrated in FIG. 3 is inserted into the flexible tube 25 and the rigid tube 26 of the connecting conduit line 24. In the rigid tube 26 of the connecting conduit line 24, the elongated section 122 of the surgical tool 120 is held in a rectilinear form.

The movement section 27 includes a main body 30 and a holding section 31. A pinion 28 interlocking with the rack 23 and a motor 29 operating the pinion 28 are provided in the main body 30. The holding section 31 holds the surgical tool 120.

The holding section 31 is coupled to be detachable from the movement section 27 and holds the proximal end of the elongated section 122 of the surgical tool 120. The holding section 31 includes a tube 32 and a connecting section 33. The tube 32 is a rigid tube which is capable of being inserted into the tube 26 of the connecting conduit line 24 and into which the elongated section 122 of the surgical tool 120 is capable of being inserted. The tube 32 is capable of being mounted using the connecting section 33.

The tube 32 holds the elongated section 122 so that the elongated section 122 of the surgical tool 120 is in a rectilinear state.

The connecting section 33 includes a through hole into which the tube 32 is inserted and which is configured to fix the tube 32 inside the through hole.

According to the present embodiment, the movement section 27 is moved to be advanced toward and retracted away from the base section 22 by a driving force of the motor 29 in a rectilinear direction in which the rack 23 is extended. Therefore, the movement section 27 is moved to closely approach or be separated from an opening 24b on the side of the proximal end of the connecting conduit line 24.

The second driving section 41 has the same configuration as the first driving section 21. The second driving section 41 includes a base section 42 and a movement section 47. The base section 42 includes a rack 43 and a connecting conduit line 44. The movement section 47 includes a main body 50 and a holding section 51. The main body 50 includes a pinion 48 and a motor 49. The second driving section 41 operates the surgical tool 120 inserted into the second proximal end tube section 9 such that the surgical tool 120 is advanced and retracted by a length which is restricted by the length between the distal end of the distal end tube section 6 and the connecting section 10.

The connecting conduit line 44 includes a tube 45 and a tube 46. The tube 45 is a flexible tube communicating with the surgical tool insertion port 15. The tube 46 is a rigid tube of which one end is connected to the flexible tube 45 and which is fixed to the base section 42. The elongated section 122 of the surgical tool 120 illustrated in FIG. 3 is inserted into the flexible tube 45 and the rigid tube 46 of the connecting conduit line 44. In the rigid tube 46 of the connecting conduit line 44, the elongated section 122 of the surgical tool 120 is configured to be held in a rectilinear form.

The holding section 51 includes a tube 52 into which the surgical tool 120 is inserted and a connecting section 53. The connecting section 53 couples the tube 52 with the base section 42.

A positional relation between the rack and the pinion in the rack-and-pinion may be reverse to the above-described positional relation. The pinions 28 and 48 and the motors 29 and 49 may be configured to be provided in the base sections 22 and 42 and the racks 23 and 43 may be provided in the movement sections 27 and 47.

The linkage section 55 includes an input unit 56, a detection unit 57, and an output unit 58. The input unit 56 receives an input from the operator via a master manipulator 154. The input from the operator via the master manipulator 154 is performed to operate the surgical tool 120. The detection unit 57 detects the position of each of the first driving section 21 and the second driving section 41. The output unit 58 supplies power for driving the motor 29 of the first driving section 21 and the motor 49 of the second driving section 41 connected to the input unit 56 and the detection unit 57.

The detection unit 57 may include a means, such as an encoder, for measuring amounts of movement of the first driving section 21 and the second driving section 41. The detection unit 57 may include a switch 65 determining whether the movement sections 27 and 47 reach predetermined positions in the base sections 22 and 42, as will be described below.

An operation input of selecting one surgical tool to be used between the surgical tool 120 mounted on the first driving section 21 and the surgical tool 120 mounted on the second driving section 41 is input from the operator to the input unit 56 via the master manipulator 154. When the operation input from the operator is input to the input unit 56, the input unit 56 acquires a positional relation between the first driving section 21 and the second driving section 41 with reference to the positions of the first driving section 21 and the second driving section 41 detected by the detection unit 57. The input unit 56 controls the output unit 58 such that the movement section 47 provided in the driving section 20 on the opposite side to the side selected through the operation input is separated from the opening on the side of the proximal end of the connecting conduit line 44. The output unit 58 drives the motor 49 in accordance with the control from the input unit 56. Further, the input unit 56 controls the output unit 58 such that the movement section 27 provided in the driving section 20 on the side selected through the operation input closely approaches the opening 24b on the side of the proximal end of the connecting conduit line 24.

Also, the input unit 56 is not capable of receiving an operation input of simultaneously using both of the surgical tool 120 mounted on the first driving section 21 and the surgical tool 120 mounted on the second driving section 41. Thus, the plurality of elongated sections 122 are not simultaneously inserted into the distal end tube section 6.

In the surgical tool holding device 1 according to the present embodiment, the input unit 56 in the linkage section 55 may be configured to receive an operation input of using none of the first driving section 21 and the second driving section 41. In this case, the linkage section 55 detects the positions of the first driving section 21 and the second driving section 41 with reference to the detection unit 57. The linkage section 55 drives the motor 29 or 49 such that the movement section 27 or 47 is separated from the opening on the side of the proximal end of the connecting conduit line 24 or 44 in the driving section (one of the first driving section 21 and the second driving section 41) which is in the closely approaching state of the movement section 27 or 47 to the opening on the side of the proximal end of the connecting conduit line 24 or 44.

Next, the configuration of the manipulator 151 will be described.

As illustrated in FIG. 1, the manipulator 151 according to the present embodiment includes the slave manipulator 152, the master manipulator 154, and a controller 157. The surgical tools 120 and the surgical tool holding device 1 are mounted on the slave manipulator 152. The master manipulator 154 is electrically connected to the slave manipulator 152 and gives an operation instruction to the slave manipulator 152. The controller 157 controls the entire medical system 150.

The surgical tool holding device 1 according to the present embodiment is configured to operate the electric pulling means 14 based on a signal sent from the slave manipulator 152 and curve the bending section 4 (see FIG. 2) electrically in response to an operation input to the master manipulator 154. The bending section 4 may be operated by an angle knob for a manual operation instead of the electric pulling means 14.

The slave manipulator 152 includes a slave arm 153, an actuator (not illustrated), and a sensor (not illustrated). At least the surgical tools 120 and the surgical tool holding device 1 described above are mounted on the slave arm 153. The actuator (not illustrated) operates the slave arm 153. The sensor (not illustrated) detects a position of the slave arm 153. The slave manipulator 152 operates the slave arm 153, the surgical tool holding device 1, and the surgical tools 120 according to an operation instruction received from the master manipulator 154.

The master manipulator 154 includes a master arm 155 and a display unit 156. The master arm 155 operates the surgical tools 120 or the surgical tool holding device 1 when an operator Op moves the master arm 155 with his or her hands. An image of a treatment target part or the like is displayed on the display unit 156.

When the master arm 155 is operated in the master manipulator 154, an operation instruction is given from the master manipulator 154. The controller 157 outputs a signal for operating the slave manipulator 152 to the slave manipulator 152 based on the foregoing operation instruction. A detailed configuration of the controller 157 will be described below.

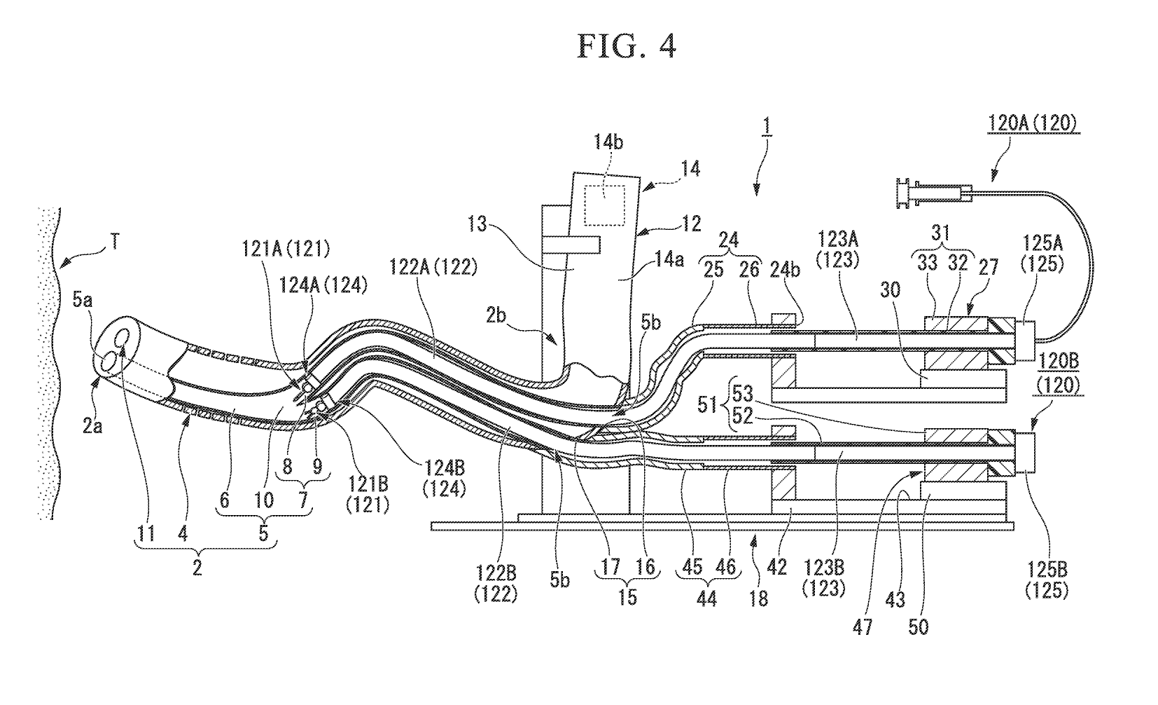

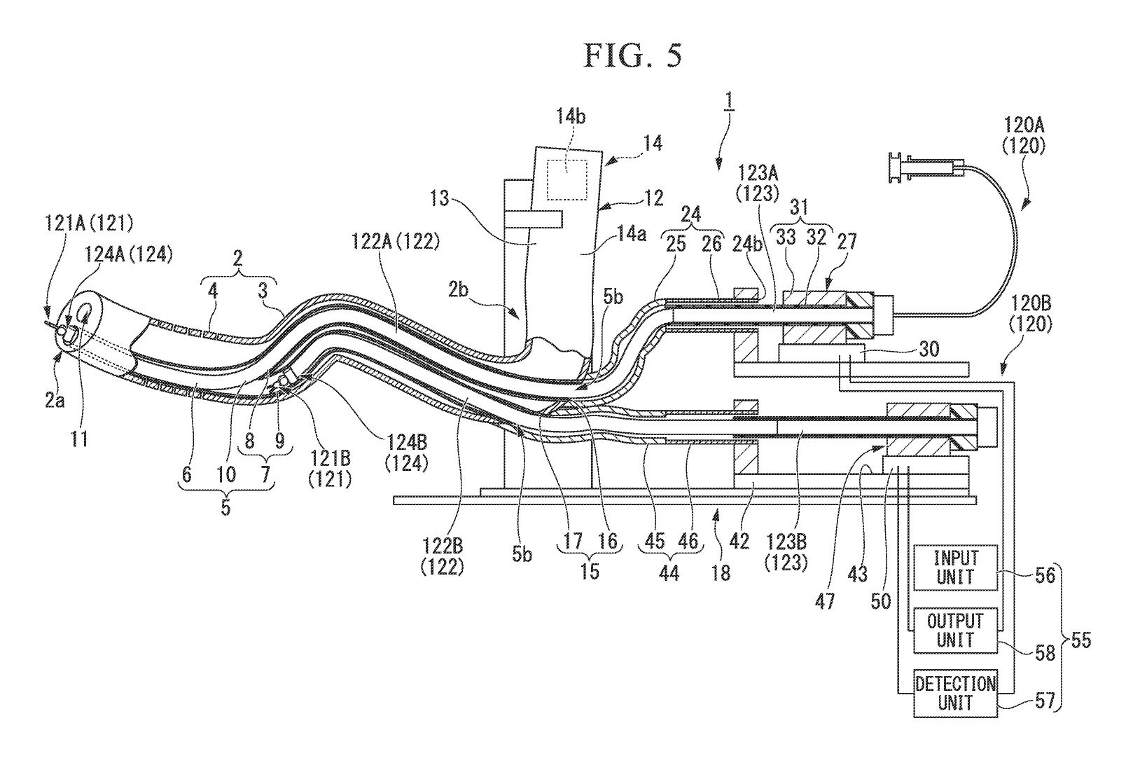

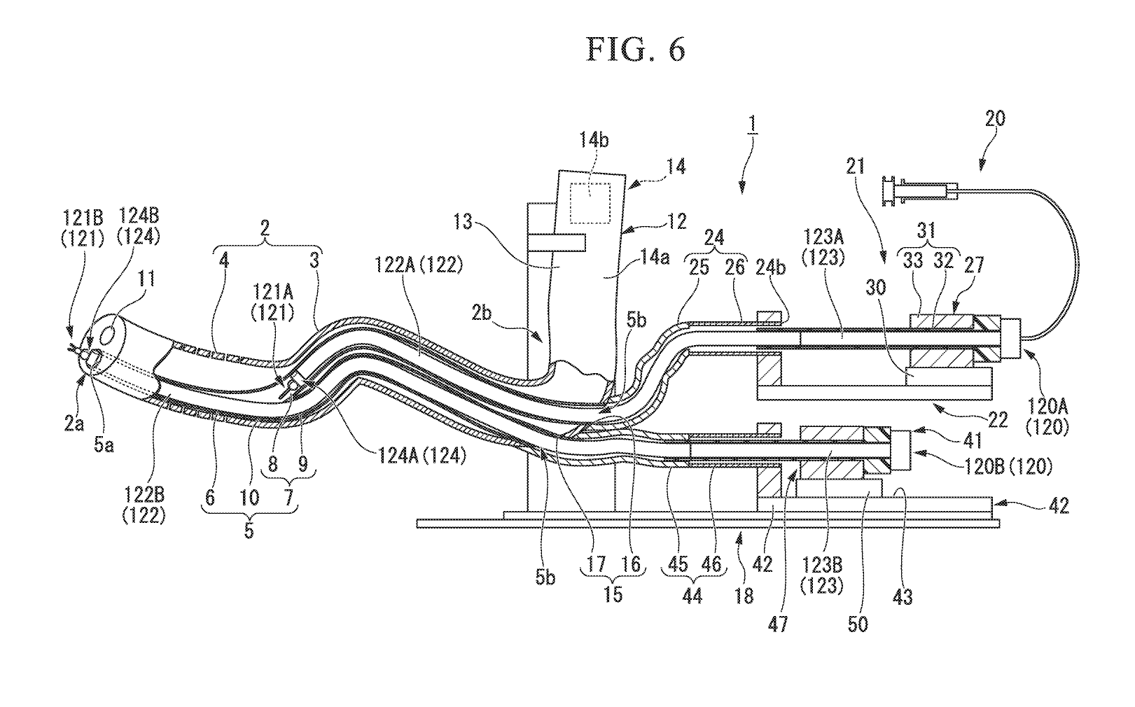

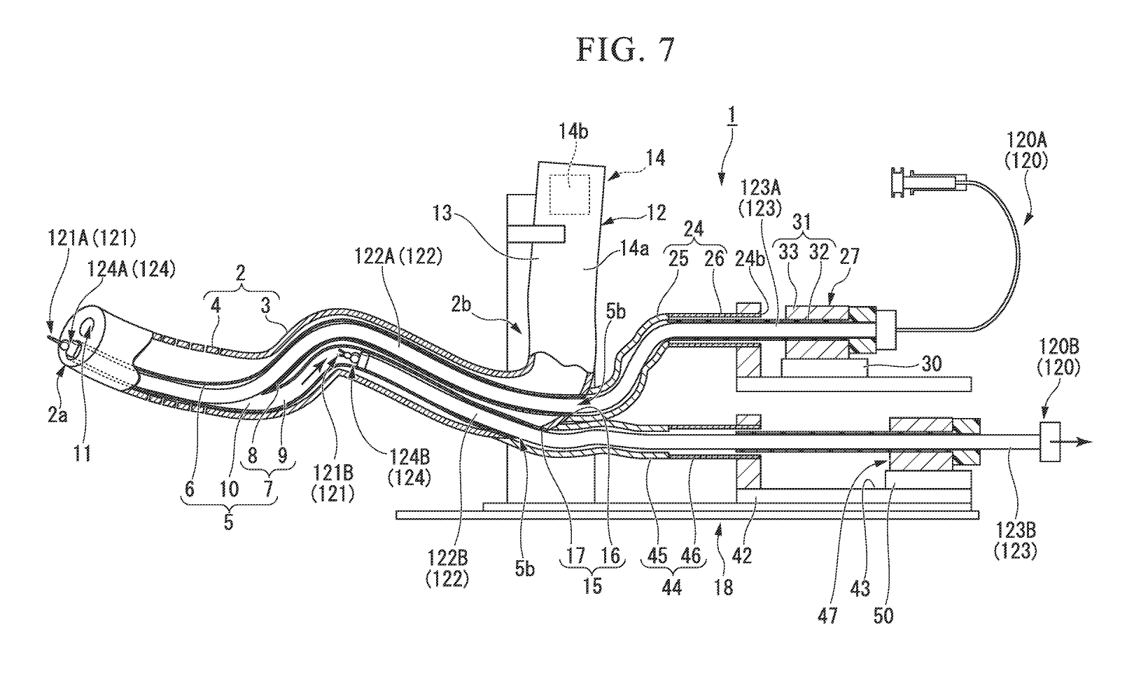

Next, operations of the medical system 150 and the surgical tool holding device 1 and the detailed configuration of the controller 157 according to the present embodiment will be described. FIG. 4 is a schematic diagram illustrating a state where the surgical tool holding device 1 according to the present embodiment is used. FIG. 5 is a schematic diagram illustrating one process when the surgical tool holding device 1 according to the present embodiment is used. FIG. 6 is a schematic diagram illustrating one process when the surgical tool holding device 1 according to the present embodiment is used. FIG. 7 is a schematic diagram illustrating one process when the surgical tool holding device 1 according to the present embodiment is used.

When the surgical tool holding device 1 is used, the insertion section 2 is first guided into a body so that the distal end 2a of the insertion section 2 is located at the position of a treatment target part T (see FIG. 4). A position and an orientation of the distal end 2a of the insertion section 2 may be adjusted to a suitable state by performing a curving operation on the bending section 4 using the electric pulling means 14 for a curving operation or a knob (not illustrated) for a curving operation, as necessary.

Subsequently, the surgical tools 120 are inserted into the first surgical tool insertion port 16 and the second surgical tool insertion port 17. When the surgical tools 120 are inserted into the first surgical tool insertion port 16 and the second surgical tool insertion port 17, the joint sections 124 of the surgical tools 120 are preferably relaxed so as to in a relaxed state that the joint sections 124 are capable of bending freely.

The foregoing surgical tool 120 for an endoscope may be mounted on only one of the first surgical tool insertion port 16 and the second surgical tool insertion port 17. In the present embodiment, a case in which the surgical tools 120 for an endoscope are inserted into both of the first surgical tool insertion port 16 and the second surgical tool insertion port 17 to be used will be described.

When the surgical tools 120 are inserted into the first surgical tool insertion port 16 and the second surgical tool insertion port 17, active curving operations of the joint sections 124 are capable of being performed by the master manipulator 154. When each surgical tool 120 is disposed inside the proximal end tube section 7, the joint section 124 is in the relaxed state. When the surgical tool 120 protrudes from the opening on the side of the distal end of the distal end tube section 6, the joint section 124 is capable of performing a curving operation in response to an operation of the operator Op on the master manipulator 154.

In the present embodiment, the joint section 124 of the surgical tool 120 is bending in response to an operation of the operator Op on the master manipulator 154 so that a treatment target part is capable of being treated at a suitable position and orientation. When one surgical tool which is being used between the two surgical tools 120 is switched with the other surgical tool which is not being used, the operator Op first performs an operation input of changing the surgical tool 120 to be used in the master manipulator 154.

As illustrated in FIG. 5, when one surgical tool 120A is selected between the two surgical tools 120 and is used in the surgical tool holding device 1, only a treatment section 121A of a first surgical tool 120A is extended from the distal end 2a of the insertion section 2. When the first treatment section 121A (which is the treatment section 121 of the surgical tool 120 inserted into the first proximal end tube section 8) is extended from the distal end 2a of the insertion section 2 through the distal end tube section 6, the treatment section 121B of a second surgical tool 120B is disposed inside the second proximal end tube section 9. When the use of the first surgical tool 120A ends, or the use of the first surgical tool 120A is interrupted and the second surgical tool 120B is switched, the operator Op performs an operation input using the input unit 56 provided in the linkage section 55 so that, as illustrated in FIG. 6, the treatment section 121A mounted on the elongated section 122A of the first surgical tool 120A is pulled back to the inside of the first proximal end tube section 8 through electric driving of the motor 29 serving as a power source and the elongated section 122B of the second surgical tool 120B is pushed into the distal end tube section 6 through electric driving of the motor 49 serving as a power source.

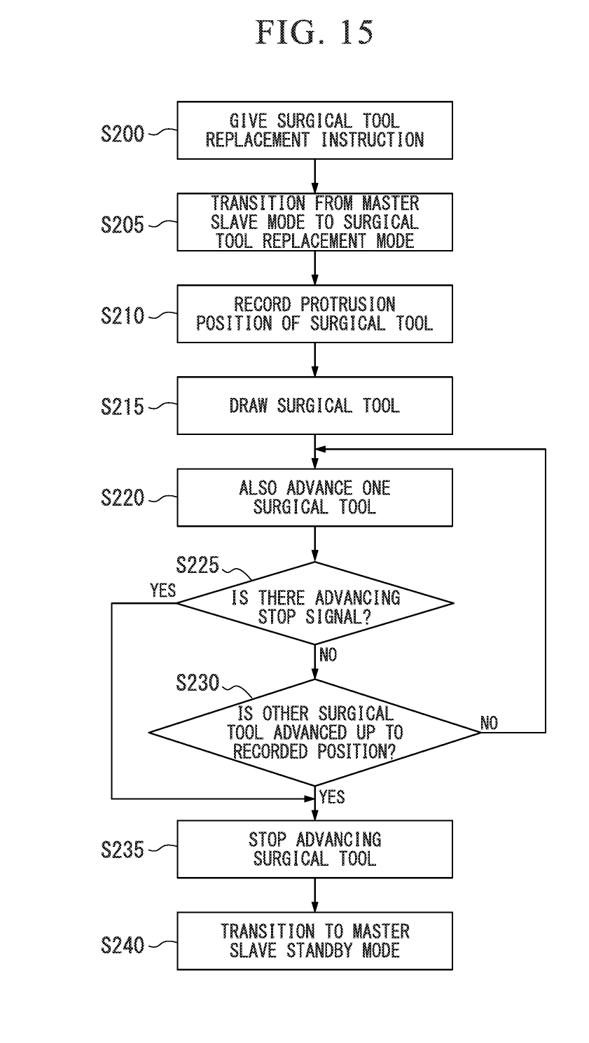

While the surgical tool is switched from the first surgical tool 120A to the second surgical tool 120B, the controller 157 first relaxes the joint section 124 of the first surgical tool 120A which is being used. That is, the electric pulling means 14 releases the pulling force applied to the bending wire connected to the joint section 124. Subsequently, the controller 157 sends a signal to the linkage section 55 of the surgical tool holding device 1 to move the movement section of the driving section 20 (the first driving section 21 or the second driving section 41) corresponding to the first surgical tool 120A which is being used to the side of the proximal end of the base section. The linkage section 55 receiving an input of the signal draws the first surgical tool 120A which was being used into the proximal end tube section 7 through the electric driving of the motor 29 serving as the power source and pushes the second surgical tool 120B into the distal end tube section 6 through the electric driving of the motor 49 serving as the power source. The controller 157 is capable of controlling the amount by which the surgical tool 120 protrudes from the distal end of the insertion section 2. For example, a protrusion position of the first surgical tool 120A which was being used is capable of being stored and a protrusion position of the second surgical tool 120B with which the first surgical tool 120A is replaced is capable of being restricted up to the foregoing stored position. A process of changing a surgical tool will be described with reference to the flowchart illustrated in FIG. 15. First, the controller 157 receives a surgical tool replacement instruction (step S200). The controller 157 receiving the surgical tool replacement instruction transitions from a master slave mode to a surgical tool replacement mode (step S205). The controller 157 having transitioned to the surgical tool replacement mode records a protrusion position of a surgical tool (step S210). Subsequently, the controller 157 draws the surgical tool 120 using the linkage section 55 (step S215). After the drawing of the first surgical tool 120A is completed in step S215, the second surgical tool 120B with which the first surgical tool 120A is replaced is advanced up to the recorded position record in the foregoing step S210 (steps S220 to S235). When the advancing of the second surgical tool 120B starts in step S220 and an advancing stop signal is subsequently input during the movement of the second surgical tool 120B, the process proceeds to step S235 through the conditional branch in step S225 and the advancing of the second surgical tool 120B is stopped. When the advancing of the second surgical tool 120B is stopped, the controller 157 transitions to a master slave standby mode (step S240). The replacement of surgical tool is capable of being realized more reliably since the controller 157 restricts the initial position at the time of the replacement of the second surgical tool 120B with which the first surgical tool 120A is replaced up to the protrusion position of the first surgical tool 120A before the replacement under an assumption that a diseased part or the like is not present up to the protrusion position of the first surgical tool 120A immediately before the replacement of the surgical tool.

Even when the master arm 155 is moved during the replacement of the surgical tool 120 in the surgical tool holding device 1, the controller 157 may cause a motion of the master arm 155 not to be transmitted to the slave manipulator 152.

When the second surgical tool 120B to be used from then on is extended (protrudes) from the distal end of the insertion section 2, the controller 157 controls the joint section 124 of the second surgical tool 120B such that the joint section 124 enters a predetermined initial orientation and is ready to receive an input from the master arm 155 by the operator Op. The operator Op can adjust (control) an amount of protrusion (an amount of advancement or retraction) with respect to the biological tissue using the second surgical tool 120B after the switching and treat a treatment target biological tissue.

When the first surgical tool 120A is being used, the joint section 124 is in a relaxed state in the second surgical tool 120B which is not used for the treatment and is pulled back into the second proximal end tube section 9 through the electric driving of the motor 49 serving as the power source. Therefore, the second surgical tool 120B is capable of being removed from the channel tube 5 by manual work or the like without conflict with the first surgical tool 120A which is being used and without conflict with an inner wall or the like of the channel tube 5, as illustrated in FIG. 7. Thereafter, even when another surgical tool (not illustrated) is mounted on the empty channel tube 5, the other surgical tool does not conflict with the first surgical tool 120A which is being used.

When a pulling force holding the joint shape is applied to the joint section 124 of the surgical tool 120, the joint section 124 is substantially a rigid section. Therefore, it may be difficult for the joint section 124 to pass through the inside of the flexible channel in some cases. According to the present embodiment, the surgical tool is capable of being replaced without any damage to the channel by relaxing the joint section 124 at a replacement timing of the surgical tool.

In FIG. 1, a treatment for an upper alimentary canal for which the surgical tool holding device 1 according to the present embodiment is inserted from a mouth is illustrated. However, the medical system 150 and the surgical tool holding device 1 according to the present embodiment are also capable of being used for a lower alimentary canal for which the surgical tool holding device 1 is inserted from an anus or in a case in which the surgical tool holding device 1 is inserted into a small incision formed in an abdominal part.

In the surgical tool holding device 1 according to the present embodiment, it is not necessary to completely remove the first surgical tool 120A and the second surgical tool 120B from the channel tube 5 when the first surgical tool 120A and the second surgical tool 120B disposed inside the channel tube 5 are switched and used. When the first surgical tool 120A is drawn to the side of the proximal end so that the treatment section 121A of the first surgical tool 120A is located on the side of the proximal end more closely than at least the connecting section 10, the treatment section 121B and the elongated section 122B of the second surgical tool 120B are extended from the distal end of the insertion section 2 via the distal end tube section 6 without conflict with the first surgical tool 120A. Further, the amount of protrusion (an amount of advancement or retraction) of the surgical tool 120 protruding from the distal end of the insertion section 2 is capable of being controlled by the driving section 20 moving the surgical tool 120. An initial position at the time of the replacement of the second surgical tool 120B with which the first surgical tool 120A is replaced is capable of being restricted up to the protrusion position of the first surgical tool 120A before the replacement based on the assumption that a diseased part or the like is not present up to the protrusion position of the first surgical tool 120A immediately before the replacement. Electric replacement of the surgical tool 120 and adjustment of the amount of advancement or retraction at the time of the protrusion of the surgical tool 120 is capable of being realized by the same translation mechanism and with a small size.

In the surgical tool holding device 1 according to the present embodiment, a movement length of the elongated section 122 for the replacement of the surgical tool 120 is short and mutual conflict between the surgical tools 120 does not occur. As a result, the surgical tool holding device 1 according to the present embodiment shortens a switching time of the plurality of surgical tools 120 and realizes excellent operability.

Also, since the connecting section 10 in the channel tube 5 is disposed inside the flexible tube section 3 and is disposed in the vicinity of the boundary between the flexible tube section 3 and the bending section 4, flexibility of the bending section 4 is high.

As a bending section provided in a known flexible endoscopic device, the bending section with which a plurality of tubular members are coupled to be rotatable and which is bending while the tubular form is held without collapse of an inner cavity of the bending section by rotation of each tubular member is known. However, when the tubular members are provided, the inner cavity of the bending section is narrower than the inner cavity of the flexible tube section in some cases. The surgical tool holding device 1 according to the present embodiment has a configuration in which one conduit line which is a surgical tool channel is present inside the bending section 4 and the conduit line is branched into two sections in the connecting section 10 located outside the bending section 4. Therefore, even when the inner cavity of the bending section 4 is narrow, the channel tube 5 is capable of being disposed inside the bending section 4 without any damage to the function of the bending section 4.

When the linkage section 55 is not provided and the two elongated sections 122 are forcedly pushed into the distal end tube section 6, the two elongated sections 122 conflict with each other. As a result, there is a probability that a treatment using the two elongated sections 122 is not capable of being performed. Also, there is a probability that the two surgical tools 120 are not capable of being extracted from the channel tube 5 of the surgical tool holding device 1.

Since the linkage section 55 is provided in the surgical tool holding device 1 according to the present embodiment, the operator Op is prevented from erroneously inserting the two elongated sections 122 of the two surgical tools 120 into the distal end tube section 6 simultaneously. When one of the two surgical tools 120 has been disposed in the distal end tube section 6, the other surgical tool 120 is prevented from being inserted into the distal end tube section 6. Since the conflict between the plurality of surgical tools 120 inside the distal end tube section 6 due to an erroneous operation of the operator Op is prevented, a risk that a treatment using the surgical tool holding device 1 being interrupted is capable of being suppressed.

The surgical tool holding device 1 according to the present embodiment can include the imaging means 11 and substantially functions as an endoscope. Therefore, it is not necessary to introduce another separate endoscope into a body to image a treatment target part, and thus it eases the patient's pain.

### Second Embodiment

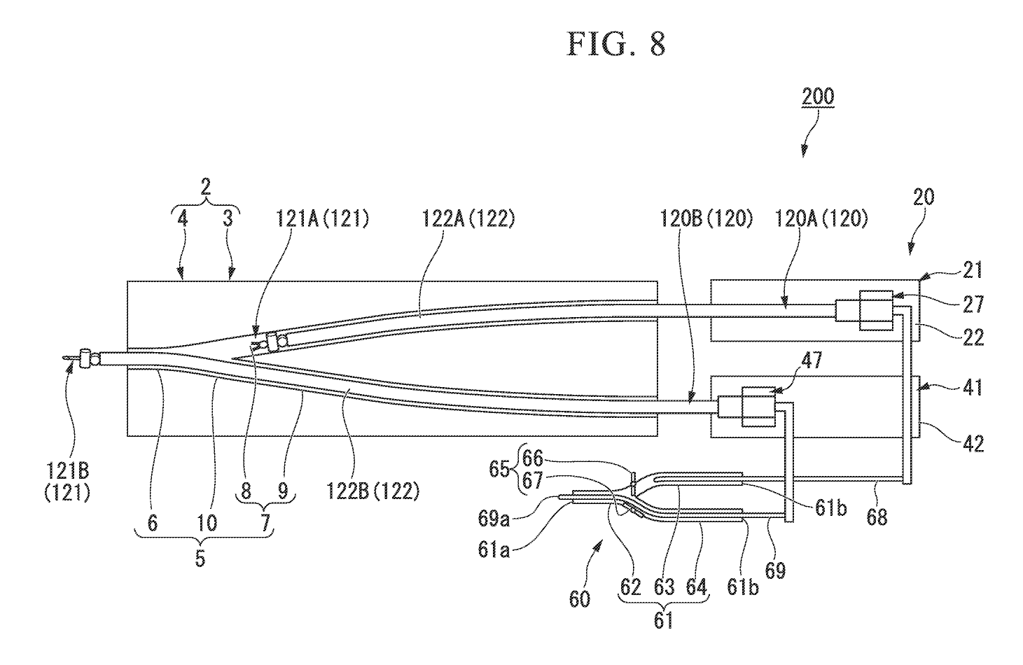

Next, a surgical tool holding device 200 and an endoscope according to a second embodiment of the present invention will be described. FIG. 8 is a schematic diagram illustrating a part of the surgical tool holding device 200 according to the second embodiment of the present invention.

As illustrated in FIG. 8, in the present embodiment, a check section 60 is provided in the driving section 20 so as to prevent the two elongated sections 122 of the two surgical tools 120 from being simultaneously inserted into the distal end tube section 6.

The check section 60 includes a stopper unit 61, a switch 65, a first sensor wire 68, and a second sensor wire 69. The stopper unit 61 is a stopper unit with a tubular shape branched into two sections. The switch 65 is disposed at the branched section of the stopper unit 61. The first sensor wire 68 is fixed to the movement section 27 of the first driving section 21 and is inserted through the stopper unit 61. The second sensor wire 69 is fixed to the movement section 47 of the second driving section 41 and is inserted through the stopper unit 61.

In the stopper unit 61, a conduit line 62 is disposed on the side of one end 61a (which is hereinafter referred to as a distal end of the stopper unit 61 and the check section 60). Also, the stopper unit 61 is branched into two sections such that two conduit lines 63 and 64 are disposed at the side of the other end 61b (which is hereinafter referred to as a proximal end of the stopper unit 61 and the check section 60). An inner diameter of the conduit line 62 disposed at the side of the distal end of the stopper unit 61 and the check section 60 is a size in which one of the first sensor wire 68 and the second sensor wire 69 is inserted and the other sensor wire is thus uninsertable.

The switch 65 is a contact switch disposed in a region on the side of the proximal end more closely than the branched section in the vicinity of the branched section of the stopper unit 61. The switch 65 includes a first switch 66 with which the first sensor wire 68 comes into contact and a second switch 67 with which the second sensor wire 69 comes into contact. The switch 65 is not limited to a contact type switch, but may be a non-contact sensor using light or magnetism.

The first sensor wire 68 is a linear elastic member which is inserted into the conduit line 63, which is the one of the two conduit lines 63 and 64 on the side of the proximal end of the stopper unit 61, from the side of the proximal end to the side of the distal end. The first sensor wire 68 operates in an integrated manner with the movement section 27 of the first driving section 21 and functions as a sensor configured to detect the position of the movement section 27. The first sensor wire 68 is disposed such that a distal end 68a of the first sensor wire 68 enters the conduit line 62 on the side of the distal end of the stopper unit 61 and occupies the inside of the conduit line 62 at a time point at which the treatment section 121 provided in the surgical tool 120 disposed inside the channel tube 5 mounted on the first driving section 21 enters the connecting section 10 of the channel tube 5 from the side of the proximal end.

The second sensor wire 69 is a linear elastic member which is inserted into the conduit line 64, which is the one other of the two conduit lines 63 and 64 on the side of the proximal end of the stopper unit 61, from the side of the proximal end to the side of the distal end. The second sensor wire 69 operates in an integrated manner with the movement section 47 of the second driving section 41 and functions as a sensor configured to detect the position of the movement section 47. The second sensor wire 69 is disposed such that a distal end 69a of the second sensor wire 69 enters the conduit line 62 on the side of the distal end of the stopper unit 61 and occupies the inside of the conduit line 62 at a time point at which the treatment section 121 provided in the surgical tool 120 disposed inside the channel tube 5 mounted on the second driving section 41 enters the connecting section 10 of the channel tube 5 from the side of the proximal end.

Next, an action of the surgical tool holding device 200 according to the present embodiment will be described.

In the surgical tool holding device 200 according to the present embodiment, by bringing the first sensor wire 68 into contact with the first switch 66, it is capable of determining that the elongated section 122A of the first surgical tool 120A mounted on the first driving section 21 is disposed inside the distal end tube section 6. In contrast, by bringing the second sensor wire 69 into contact with the second switch 67, it is capable of determining that the elongated section 122B of the second surgical tool 120B mounted on the second driving section 41 is disposed inside the distal end tube section 6.

Further, only one of the first sensor wire 68 and the second sensor wire 69 is configured to be capable of being inserted into the conduit line 62 on the side of the distal end of the stopper unit 61. Even when the elongated section 122A of the first driving section 21 and the elongated section 122B of the second driving section 41 are pushed together into the distal end tube section 6 due to an erroneous operation, the first sensor wire 68 and the second sensor wire 69 conflict with each other in the branched section of the conduit lines 62, 63, and 64 in the stopper unit 61. Before the treatment section 121A of the first surgical tool 120A and the treatment section 121B of the second surgical tool 120B completely enter the connecting section 10, operations of the first driving section 21 and the second driving section 41 are mechanically restricted.

According to the surgical tool holding device 200 according to the present embodiment, it is capable of mechanically restricting the simultaneous insertion of the plurality of elongated sections 122A and 122B into the distal end tube section 6 in the channel tube 5 so that the surgical tool holding device 200 has a simple and highly reliable configuration.

### Third Embodiment

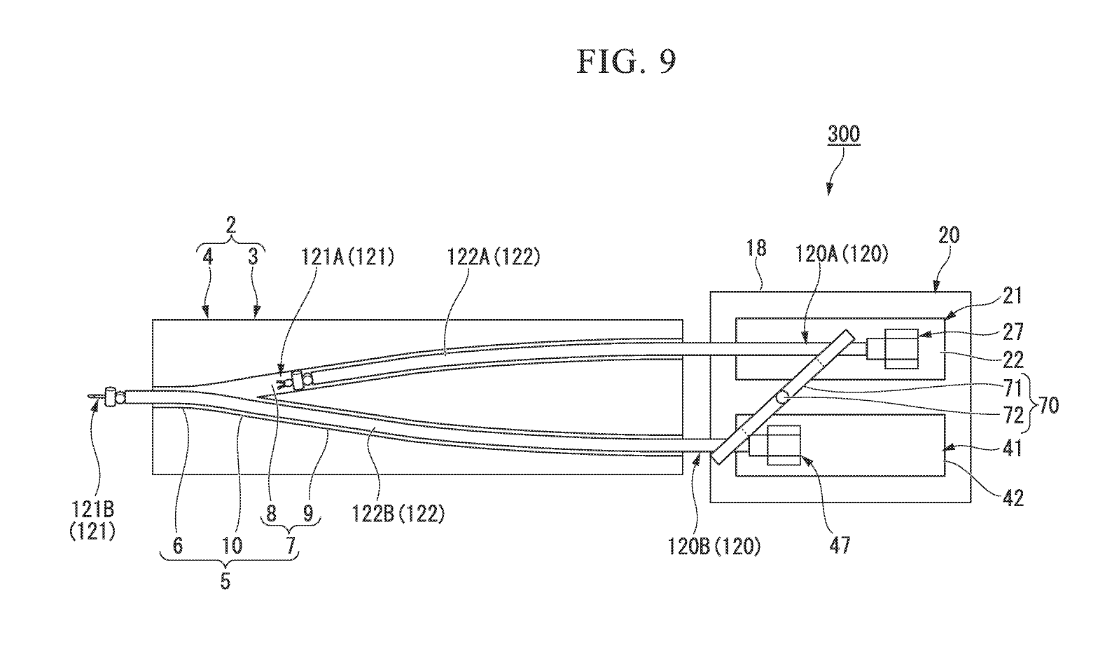

Next, a surgical tool holding device 300 and an endoscope according to a third embodiment of the present invention will be described. FIG. 9 is a schematic diagram illustrating a part of the surgical tool holding device 300 according to the third embodiment of the present invention.

As illustrated in FIG. 9, the surgical tool holding device 300 according to the third embodiment includes a check section 70 having a configuration different from the check section 60 described in the above-described embodiment.

The check section 70 includes a switch 71 and a supporting section 72. The switch 71 is a bar-like switch which has a rotational center in the middle of the first driving section 21 and the second driving section 41. The switch 71 is capable of coming into contact with the movement section 27 of the first driving section 21 and the movement section 47 of the second driving section 41. The supporting section 72 is configured such that the switch 71 is rotatable about the rotational center and supports the switch 71.

In the surgical tool holding device 300 according to the third embodiment, the first driving section 21 and the second driving section 41 are mounted on the base 18 so that a movable direction of the movement section 27 of the first driving section 21 and a movable direction of the movement section 47 of the second driving section 41 are parallel to each other.

The supporting section 72 is fixed to the base 18. The position of the supporting section 72 with respect to the first driving section 21 and the second driving section 41 is set such that the movement section 27 of the first driving section 21 and the movement section 47 of the second driving section 41 together come into contact with the switch 71 when the treatment section 121A of the first surgical tool 120A and the treatment section 121B of the second surgical tool 120B are located slightly closer on the side of the proximal end tube section 7 from the connecting section 10.

In the surgical tool holding device 300 according to the third embodiment, even when the movement section 27 of the first driving section 21 and the movement section 47 of the second driving section 41 operate to press the switch 71 simultaneously due to an erroneous operation, one of the treatment section 121A of the first surgical tool 120A and the treatment section 121B of the second surgical tool 120B is inevitably in a state drawn into the proximal end tube section 7. Thus, according to the present embodiment, conflict between the elongated sections 122A and 122B in the distal end tube section 6 does not occur.

Also, according to the direction in which the switch 71 is rotated about the rotation center to be pressed, it is capable of determining whether one of the first surgical tool 120A mounted on the first driving section 21 and the second surgical tool 120B mounted on the second driving section 41 is inserted into the distal end tube section 6 to be in use.

### Fourth Embodiment

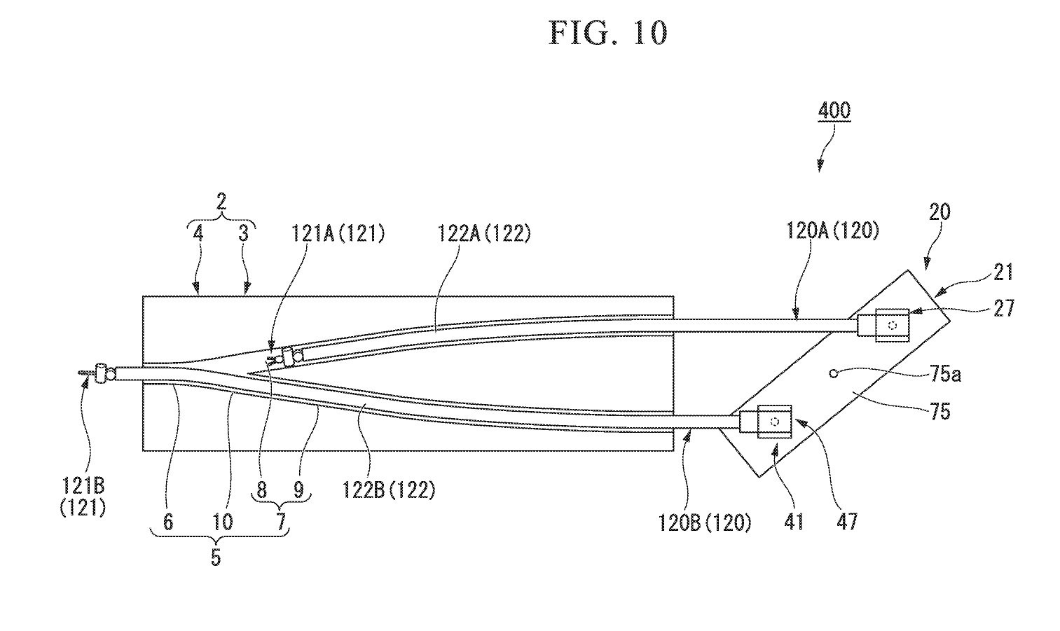

Next, a surgical tool holding device 400 and an endoscope according to a fourth embodiment of the present invention will be described. FIG. 10 is a schematic diagram illustrating a part of the surgical tool holding device 400 according to the fourth embodiment of the present invention.

As illustrated in FIG. 10, the surgical tool holding device 400 according to the fourth embodiment includes a coupling member 75 coupling the movement section 27 of the first driving section 21 with the movement section 47 of the second driving section 41 instead of the above-described linkage section 55. The coupling member 75 includes a rotational center 75a between the first driving section 21 and the second driving section 41.

In the surgical tool holding device 400 according to the present embodiment, one of the first driving section 21 and the second driving section 41 may actively operate. For example, when driving means configured by a rack-and-pinion and a motor is installed in the first driving section 21, the second driving section 41 may be a rail or the like that rectilinearly moves the movement section 47 relative to the base section 42.

In the surgical tool holding device 400 according to the present embodiment, operations of the movement section 27 of the first driving section 21 and the movement section 47 of the second driving section 41 are always restricted by the coupling member 75 different from the above-described third embodiment. When the movement section 27 of the first driving section 21 operates to push the elongated section 122A into the side of the distal end of the channel tube 5, the movement section 47 of the second driving section 41 operates in the following manner to draw the elongated section 122B to the side of the proximal end of the channel tube 5.

When a means for directly rotating the coupling member 75 about the rotational center 75a is provided, the means for directly driving the first driving section 21 and the second driving section 41 is not necessary.

### Fifth Embodiment

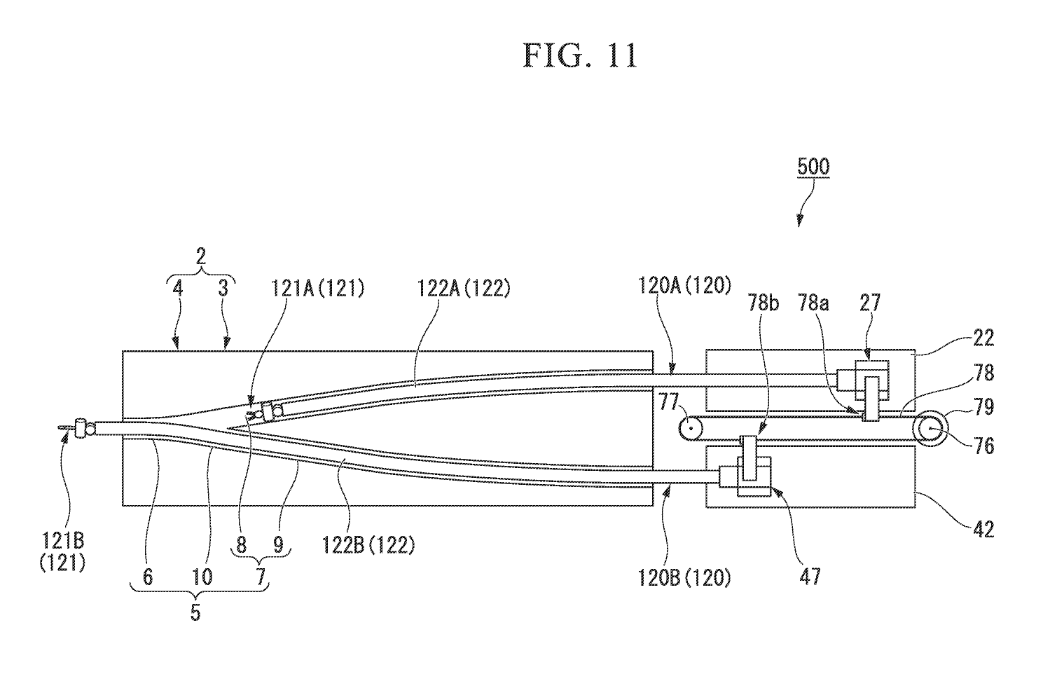

Next, a surgical tool holding device 500 and an endoscope according to a fifth embodiment of the present invention will be described. FIG. 11 is a schematic diagram illustrating a part of the surgical tool holding device 500 according to the fifth embodiment of the present invention.

In the present embodiment, as illustrated in FIG. 11, a pair of pulleys 76 and 77, a wire 78, and a motor 79 are included instead of the coupling member 75 described in the above-described fourth embodiment. The wire 78 is a series of lopped wires suspended around the pulleys 76 and 77. The motor 79 rotates one (the pulley 76 in the present embodiment) of the pair of pulleys 76 and 77.

The first driving section 21 and the second driving section 41 do not include a driving means configured by a rack-and-pinion and a motor moving the movement sections 27 and 47 with respect to the base sections 22 and 42. The first driving section 21 and the second driving section 41 are configured such that the movement sections 27 and 47 are advanced and retracted with respect to the base sections 22 and 42 along a rail or the like by the rail. Also, according to the present embodiment, a movable direction of the movement section 27 of the first driving section 21 and a movable direction of the movement section 47 of the second driving section 41 are mutually parallel.

In the surgical tool holding device 500 according to the present embodiment, the movement section 27 of the first driving section 21 and the movement section 47 of the second driving section 41 are advanced and retracted by a driving force of the motor 79 rotating the pair of pulleys 76 and 77. The pair of pulleys 76 and 77 are disposed between the first driving section 21 and the second driving section 41, and the wire 78 is suspended around the pair of pulleys. The wire 78 is disposed in an elliptical form with a major axis in the movable directions of the movement section 27 of the first driving section 21 and the movement section 47 of the second driving section 41.

In the wire 78 suspended around the pair of pulleys 76 and 77, a section (indicated by reference numeral 78a) located closer to the first driving section 21 is coupled with the movement section 27 of the first driving section 21. Also, in the wire 78 suspended around the pair of pulleys 76 and 77, a section (indicated by reference numeral 78b) located closer to the second driving section 41 is coupled with the movement section 47 of the second driving section 41.

In the surgical tool holding device 500 according to the present embodiment, when the pair of pulleys 76 and 77 are rotated by the motor 79, a section closer to the first driving section 21 and a section closer to the second driving section 41 in the wire 78 suspended around the pulleys 76 and 77 are moved in opposite directions. Therefore, when the pair of pulleys 76 and 77 are rotated and one of the first driving section 21 and the second driving section 41 pushes one of the first surgical tool 120A and the second surgical tool 120B into the distal end tube section 6, the other of the first driving section 21 and the second driving section 41 draws the other of the first surgical tool 120A and the second surgical tool 120B toward the proximal end tube section 7.

Thus, in the surgical tool holding device 500 according to the present embodiment, the treatment sections 121 and the elongated sections 122 of two surgical tools 120 do not conflict with each other inside the distal end tube section 6, as in the above-described first embodiment.

### Sixth Embodiment

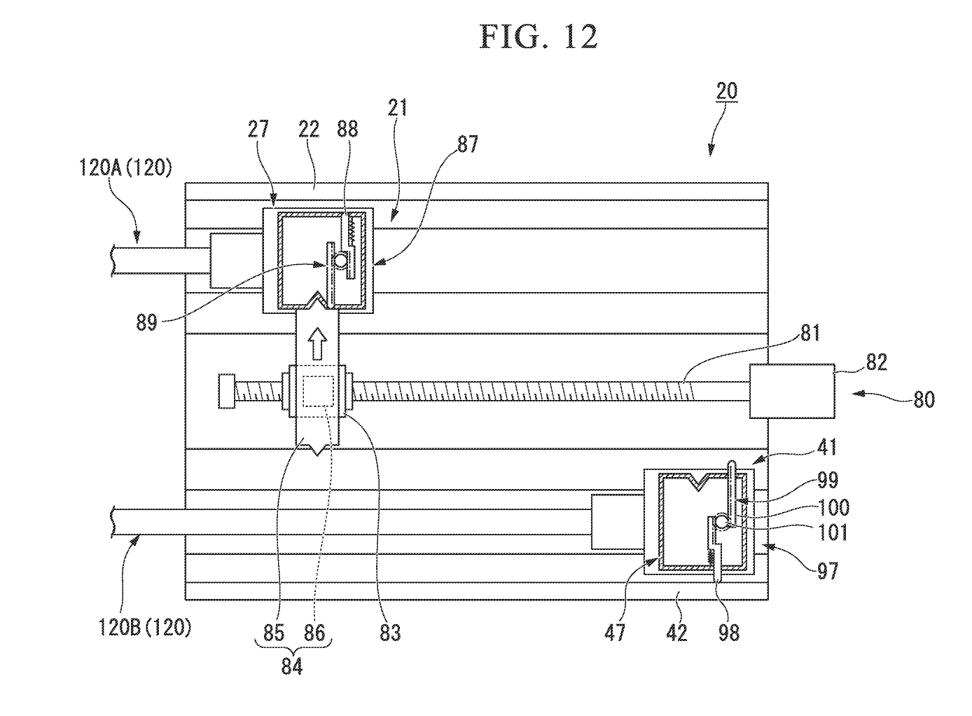

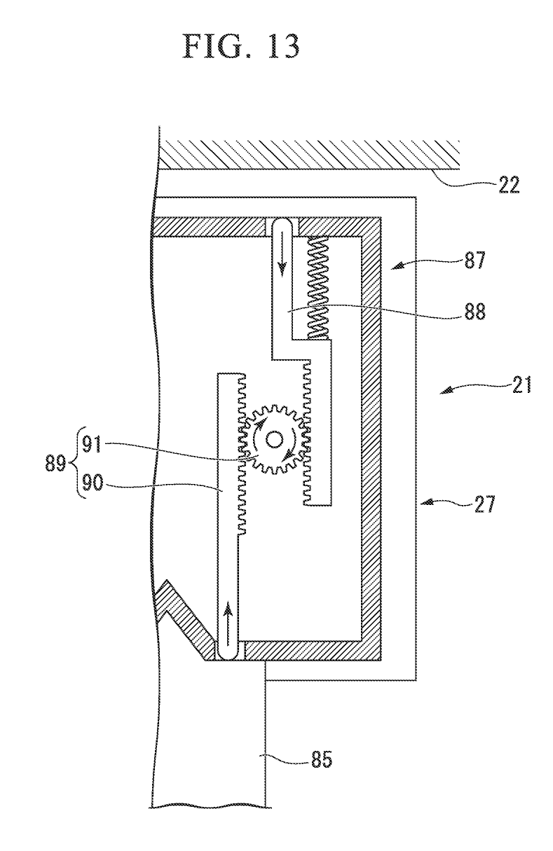

Next, a surgical tool holding device 1 and an endoscope according to a sixth embodiment of the present invention will be described. FIG. 12 is a schematic diagram illustrating a part of the surgical tool holding device 1 according to the sixth embodiment of the present invention. FIG. 13 is a schematically expanded view illustrating a part of the surgical tool holding device 1 according to the present embodiment.

In the surgical tool holding device 1 according to the present embodiment, the first driving section 21 and the second driving section 41 freely operate along a rail or the like as in the above-described fifth embodiment. As illustrated in FIG. 12, a driving means 80 for selecting and operating the movement section 27 of the first driving section 21 or the movement section 47 of the second driving section 41 is provided. Also, stoppers 87 and 97 individually fixing the positions of the movement sections 27 and 47 to the base sections 22 and 42 are provided in the movement section 27 of the first driving section 21 and the movement section 47 of the second driving section 41, respectively.

The driving means 80 according to the present embodiment includes a screw 81, a motor 82, a nut section 83, and a switching unit 84. The screw 81 is a bar-like screw that extends in a direction parallel to the movable directions of the movement section 27 of the first driving section 21 and the movement section 47 of the second driving section 41. The motor 82 rotates the screw about a center line of the screw. The nut section 83 is fitted into the screw 81. The switching unit 84 is installed in the nut section 83 and is selectively coupled with the movement sections 27 and 47.

The switching unit 84 includes a movement body 85 and an actuator 86. The movement body 85 includes an uneven section that moves to the nut section 83 in a direction perpendicular to the center line of the screw 81 and engages with the movement sections 27 and 47. The actuator 86 moves the movement body 85 relative to the nut section 83. Also, in the present embodiment, an uneven section engaging with the movement body 85 of the switching unit 84 is provided in each of the movement section 27 of the first driving section 21 and the movement section 47 of the second driving section 41.

As illustrated in FIG. 13, the stopper 87 provided in the movement section 27 of the first driving section 21 includes a stopper pin 88 and a releasing mechanism 89. The stopper pin 88 is biased by a coil spring or the like to come into contact with the base section 22. The releasing mechanism 89 separates the stopper pin 88 from the base section 22 when the movement body 85 comes into contact with the releasing mechanism 89. According to the present embodiment, the releasing mechanism 89 includes a releasing pin 90 with which the movement body 85 comes into contact and a gear 91 transmitting an operation of the releasing pin 90 to the stopper pin 88. The stopper pin 88 and the releasing pin 90 are each supported by the movement section 27 to as to be movable in parallel. Racks are formed in the stopper pin 88 and the releasing pin 90. The gear 91 of the releasing mechanism 89 meshes with the racks of the stopper pin 88 and the releasing pin 90 so that the stopper pin 88 and the releasing pin 90 move in opposite directions.

The stopper 97 provided in the movement section 47 of the second driving section 41 has the same configuration as the stopper 87 provided in the movement section 27 of the first driving section 21. The stopper 97 provided in the movement section 47 of the second driving section 41 includes a stopper pin 98, a releasing pin 100, and a releasing mechanism 99 including a gear 101.

In the surgical tool holding device 1 according to the present embodiment, the nut section 83 is moved by rotating the screw 81. Accordingly, the movement body 85 is conveyed to one of the movement section 27 of the first driving section 21 and the movement section 47 of the second driving section 41 in the center line direction of the screw 81. Further, the actuator 86 of the switching unit 84 moves the movement body 85 so that the uneven section of the movement body 85 engages with the uneven section of one of the movement section 27 of the first driving section 21 and the movement section 47 of the second driving section 41.

When the movement body 85 engages with the movement section 27 of the first driving section 21, the stopper pin 88 is separated from the base section 22 in the stopper 87 so that the movement section 27 of the first driving section 21 is capable of moving relative to the base section 22.

When the movement body 85 engages with the movement section 47 of the second driving section 41, the stopper pin 98 is separated from the base section 42 in the stopper 97 so that the movement section 47 of the second driving section 41 can move relative to the base section 42.

In the surgical tool holding device 1 according to the present embodiment, one movement section selected between the movement section 27 of the first driving section 21 and the movement section 47 of the second driving section 41 is capable of being operated by the driving means 80 and the unselected other movement section is not operated.

When the screw 81 is rotated about the center line in this state, the nut section 83 is advanced and retracted in the center line direction of the screw 81 and the movement section engaging with the movement body 85 between the movement sections 27 and 47 is advanced and retracted in the center line direction of the screw 81 in an integrated manner with the nut section 83.

With the configuration according to the present embodiment, the same advantages as in the above-described first embodiment can be obtained.

### Seventh Embodiment

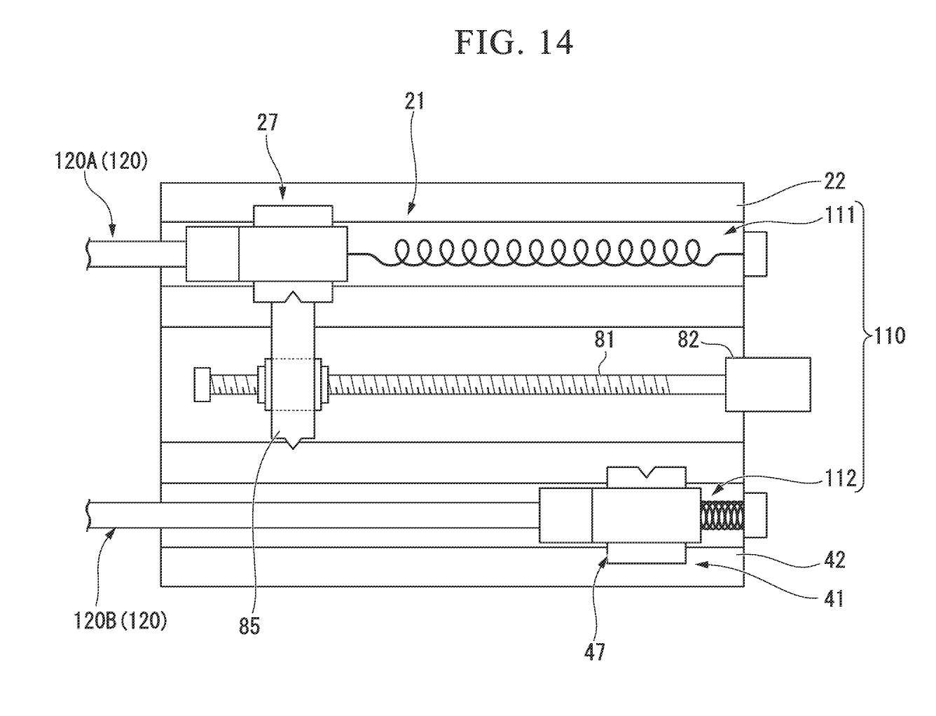

Next, a surgical tool holding device 1 and an endoscope according to a seventh embodiment of the present invention will be described. FIG. 14 is a schematic diagram illustrating a part of the surgical tool holding device 1 according to the seventh embodiment of the present invention.

As illustrated in FIG. 14, the surgical tool holding device 1 according to the present embodiment includes a pulling means 110 for pulling the surgical tool 120 back to the side of the proximal end instead of the stoppers 87 and 97 described in the above-described sixth embodiment.

In the surgical tool holding device 1 according to the present embodiment, the pulling means 110 includes pullback coil springs 111 and 112. One end of the pullback coil spring 111 is fixed to the movement section 27 of the first driving section 21 and the other end thereof is fixed to the base section 22 of the first driving section 21. One end of the pullback coil spring 112 is fixed to the movement section 47 of the second driving section 41 and the other end thereof is fixed to the base section 42 of the second driving section 41.

In the surgical tool holding device 1 according to the present embodiment, when the movement body 85 described in the foregoing sixth embodiment is separated from the movement section 27 of the first driving section 21 or the movement section 47 of the second driving section 41, the movement section 27 or 47 released from the engagement with the movement body 85 is moved toward the side of the proximal end by the pullback coil spring 111 or 112. In the present embodiment, the movement body 85 selectively engages with only one of the movement section 27 of the first driving section 21 and the movement section 47 of the second driving section 41. In the surgical tool holding device 1 according to the present embodiment, since the movement body 85 does not simultaneously engage with the movement section 27 of the first driving section 21 and the movement section 47 of the second driving section 41, the movement section of one of the first driving section 21 and the second driving section 41 is normally located at the proximal end of the base section.

With the configuration according to the present embodiment, the treatment sections 121 and the elongated sections 122 of two surgical tools 120 are prevented from interfering with each other as in each of the above-described embodiments.

In the foregoing embodiments, the imaging means 11 may not be provided in the surgical tool holding device 1. That is, the surgical tool holding device 1 may not include an examining or imaging function of a general endoscopic device. The surgical tool holding device 1 including no imaging means 11 is preferably configured such that the position of each surgical tool 120 held in the surgical tool holding device 1 is capable of being realized visually by a user. For example, the surgical tool holding device 1 including no imaging means 11 is preferably used along with an observation means such as a known endoscopic device or ultrasonic endoscope.

The preferred embodiments of the present invention have been described above, but the present invention is not limited to these embodiments. Additions, omissions, substitutions, and other changes of the configurations can be made within the scope of the present invention without departing from the gist of the present invention. The present invention is not limited to the foregoing description, but is limited only to the scope of the appended claims.

1. A surgical tool holding device that holds a plurality of surgical tools comprising:
a flexible insertion section having a distal end and a proximal end, and being configured such that a surgical tool channel into which the plurality of surgical tools are inserted is formed;
at least a driving section configured to advance and retract each of the plurality of surgical tools inside the surgical tool channel;
a operating section configured to manipulate the plurality of surgical tools; and
a controller configured to control an amount of protrusion of the surgical tool protruding from a distal end of the surgical tool channel, from the surgical tool channel,
wherein the insertion section includes:
a distal end tube section configured to be one conduit line including an opening on a side of the distal end of the insertion section;
a first proximal end tube section configured to be one conduit line including an opening on a side of the proximal end of the insertion section;
a second proximal end tube section configured to be one conduit line including an opening on the side of the proximal end of the insertion section; and
a connecting section in which the distal end tube section, the first proximal end tube section, and the second proximal end tube section are connected and disposed inside the insertion section when the distal end tube section, the first proximal end tube section, and the second proximal end tube section communicate with each other.

2. The surgical tool holding device according to claim 1, wherein the insertion section further includes:
a flexible tube section; and
a bending section configured to be disposed on the side of the distal end of the insertion section, connected with the flexible tube section, and operated to perform a bending operation,
wherein the connecting section is disposed inside the flexible tube section and near a boundary between the flexible tube section and the bending section.

3. The surgical tool holding device according to claim 1, wherein the driving section holds a part of a section of the surgical tool extended from the side of the proximal end of the surgical tool channel to an outside of the insertion section, and advances and retracts a section of the surgical tool inserted into the surgical tool channel in a conduit line central axis direction of the surgical tool channel by advancing and retracting the held section of the surgical tool.

4. The surgical tool holding device according to claim 1, wherein the driving section includes:
a first driving section configured to advance and retract the surgical tool inserted into the first proximal end tube section only by a length between a distal end of the distal end tube section and the connecting section;
a second driving section configured to advance and retract the surgical tool inserted into the second proximal end tube section only by the length between the distal end of the distal end tube section and the connecting section; and
a linkage section configured to cause the first and second driving sections to cooperatively operate such that the surgical tool inserted into the second proximal end tube section is drawn and held into the second proximal end tube section just before the connecting section when the surgical tool inserted into the first proximal end tube section is located inside the distal end tube section and such that the surgical tool inserted into the first proximal end tube section is drawn and held into the first proximal end tube section just before the connecting section when the surgical tool inserted into the second proximal end tube section is located inside the distal end tube section.

5. The surgical tool holding device according to claim 1,
wherein a shape of a cross-section perpendicular to a central axis of the distal end tube section, a shape of a cross-section perpendicular to a central axis of the first proximal end tube section, and a shape of a cross-section perpendicular to a central axis of the second proximal end tube section are substantially the same, and
wherein an area of the cross-section perpendicular to the central axis of the distal end tube section, an area of the cross-section perpendicular to the central axis of the first proximal end tube section, and an area of the cross-section perpendicular to the central axis of the second proximal end tube section are substantially the same.

6. The surgical tool holding device according to claim 5,
wherein each of the plurality of surgical tools includes a treatment section configured to perform a treatment on a treatment target and an elongated section configured to be coupled with the treatment section and inserted into the surgical tool channel, and
wherein the distal end tube section is a tube with a dimension in which the treatment section and the elongated section of only one of the plurality of surgical tools are insertable and the treatment sections and the elongated sections of two or more of the plurality of surgical tools conflict with each other when the treatment sections and the elongated sections are simultaneously inserted.

7. The surgical tool holding device according to claim 1,
wherein the plurality of surgical tools include a first surgical tool and a second surgical tool, and
wherein the controller records an amount of protrusion of the first surgical tool protruding from the distal end of the surgical tool channel and restricts an amount of protrusion of the second surgical tool protruding from the surgical tool channel so that the second surgical tool protrudes only up to the recorded amount of protrusion when the first surgical tool protruding from the distal end is drawn and the second surgical tool is caused to protrude from the distal end of the surgical tool channel.

8. An endoscope comprising:
the surgical tool holding device according to claim 1; and
an imaging means configured in the insertion section and configured to image the treatment target.

9. A medical system comprising:
the surgical tool holding device according to claim 1;
a plurality of surgical tools configured to perform a treatment on a treatment target; and
a manipulator configured to operate the surgical tool holding device and the plurality of surgical tools,
wherein each of the plurality of surgical tools includes
a treatment section configured to perform the treatment on the treatment target;
an elongated section configured to be coupled with the treatment section and inserted into the surgical tool channel;
a joint section configured to couple the treatment section with the elongated section and change an orientation of the treatment section with respect to the elongated section; and
a wire configured to be disposed inside the elongated section and transmit a force to operate the joint section,
wherein the manipulator includes
a master manipulator configured to receive an operation input from an operator;
a slave manipulator configured to be electrically connected to the master manipulator and operate the wire in accordance with the operation input to the master manipulator; and
a controller configured to be electrically connected to the master manipulator and the slave manipulator and control an orientation of the joint section,
wherein, when the joint section is extended from the distal end tube conduit and a halt command of the surgical tool including the joint section is performed on the manipulator, the controller generates a signal for manipulating the wire so that the joint section is relaxed and outputs the signal to the slave manipulator, and
wherein, when the joint section is relaxed in response to the signal, the controller outputs, to the slave manipulator, a signal for drawing the treatment section and the elongated section until the treatment section is located inside the first or second proximal end tube section.
